(19) United States
(12) Patent Application Publication
(10) Pub. No.: US 2007/0179468 A1
(43) Pub. Date: Aug. 2, 2007

(54) PET URINE PAD WITH VERTICAL TARGET AND ABSORBENT BASE

(76) Inventor: Jeffery LaBelle, Green Cove Springs, FL (US)

Correspondence Address: Mark James Young, 12086 Fort Caroline Rd., Unit 202, Jacksonville, FL, 32225, US

(21) Appl. No.: 11/307,257

(22) Filed: Jan. 30, 2006

(51) Int. Cl. A61F 13/15

(52) U.S. Cl. 604/385.19; 604/385.01

(57) ABSTRACT

A urine pad includes a vertical target and a base. The vertical target is substantially erect to provide an object with 360° accessibility conducive to spraying from any angle. The base surrounds the vertical target and provides a padded structure for absorbing urine. Urine sprayed onto the vertical target is substantially absorbed into the target. Excess urine runs downward and becomes absorbed into the base. The base and vertical target are comprised of a laminate, including an impermeable layer, a permeable topsheet layer and an absorbent layer sandwiched between the impermeable and permeable topsheet layers. The vertical target may be comprised of a pocket attached to or formed in the horizontal base, with a support object disposed within the pocket to maintain the pocket erect.

### FIELD OF THE INVENTION

This invention relates to pets, and more particularly, to a urine pad having a vertical target and an absorbent base.

### BACKGROUND

There are many ways to potty train a pet. Some pet owners utilize pads which they encourage their pet to use for going to the bathroom. Many such pads are scented with a chemical that attracts the pet to use them. While such pads are useful for providing an absorbent surface for collecting urine and fecal matter, they are ineffective in addressing the problem of spraying.

Animals such as dogs frequently spray urine against a vertical structure. This activity, known as “spraying” or “marking,” is a commonplace instinctive method of identifying and protecting a territory. When performed outdoors, spraying goes unnoticed. However, indoors, animal spraying can be a serious problem, particularly in households where pets are expected to urinate on pads. As conventional pads lack a vertical structure, the spray typically marks adjacent walls and/or furniture. Left untreated, the sprayed urine will severely stain and deteriorate the targeted structure and create an extremely unpleasant stench.

To remediate indoor spraying problems, pet owners have tried many different solutions. Some pet owners repeatedly clean and deodorize the targeted areas. Others may alter (e.g., sterilize) or medicate their dog to prevent the behavior. Yet other pet owners may resort to shielding frequently targeted areas. Occasionally, dogs are forced to wear diapers. Frustrated owners may eventually severely confine or even part with their dog. While some of the foregoing techniques are useful in the short-term to varying degrees, each has limited effectiveness and desirability as a long-term solution. Thus, there remains a need for controlling the spraying of urine by a dog, without harming the dog or diminishing its quality of life.

The invention is directed to overcoming one or more of the problems as set forth above.

### SUMMARY

To overcome problems as set forth above, one aspect of an exemplary embodiment of a urine pad according to the principles of the invention resides in a pad comprising a vertical target and a base. The vertical target is positioned substantially erect to provide an object with 360° accessibility conducive to spraying from any angle. The base surrounds the vertical target and provides a padded structure for absorbing urine. Urine sprayed onto the vertical target is substantially absorbed into the target. Excess urine runs downward and becomes absorbed into the base.

An exemplary base and vertical target are comprised of a laminate, including an impermeable layer, an absorbent layer and a permeable topsheet layer. The absorbent layer is disposed between the impermeable and permeable topsheet layers.

In one embodiment, the vertical target is comprised of a pocket attached to or formed in the horizontal base. A support object such as a plastic bottle may be disposed within the pocket to maintain the pocket erect. The vertical target may be removably or permanently attached to the horizontal base, near the center of the horizontal base.

Thus, the invention provides an absorbent urine pad with a vertical target conducive to spraying by an animal.

### BRIEF DESCRIPTION OF THE DRAWINGS

The foregoing and other aspects, objects, features and advantages of the invention will become better understood with reference to the following description, appended claims, and accompanying drawing, where:

FIG. 1 provides a high level perspective view that conceptually illustrates a urine pad in accordance with the principles of the invention; and

FIG. 2 provides a high level perspective view that conceptually illustrates a urine pad and a vertical support structure in accordance with the principles of the invention; and

FIG. 3 conceptually illustrates top plan view of a urine pad in accordance with principles of the invention; and

FIG. 4 conceptually illustrates side section view of a portion of a urine pad in accordance with principles of the invention; and

FIG. 5A provides a perspective view of an exemplary support holder for a urine pad in accordance with principles of the invention; and

FIG. 5B provides a high level perspective view that conceptually illustrates a urine pad and a vertical support structure with a support holder in accordance with the principles of the invention.

The Figures are provided to conceptually illustrate exemplary embodiments in accordance with principles of the invention. However, the invention is not limited to those exemplary embodiments depicted in the Figures. Those skilled in the art will appreciate that the Figures are not intended to be drawn to any particular scale; the invention is not limited to the dimensions or proportions shown in Figures; the invention is also not limited to the selection, arrangement and coordination of information, items, aesthetic elements, and components shown in the Figures; and the Figures are not intended to illustrate every embodiment of the invention.

### DETAILED DESCRIPTION

This invention provides a urine pad having a vertical target and a base. The vertical target is positioned substantially erect to provide an object conducive to spraying from any angle. The base surrounds the vertical target and provides a padded structure for absorbing urine. Urine sprayed onto the vertical target is substantially absorbed into the target. Excess urine runs downward and becomes absorbed into the base.

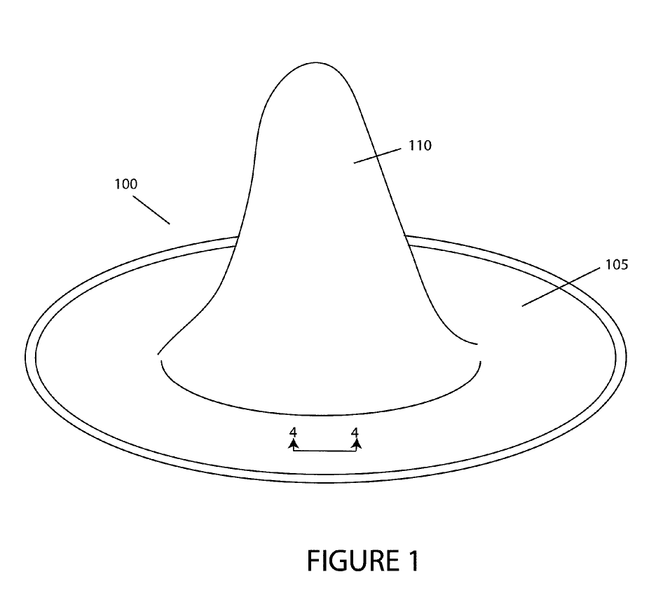

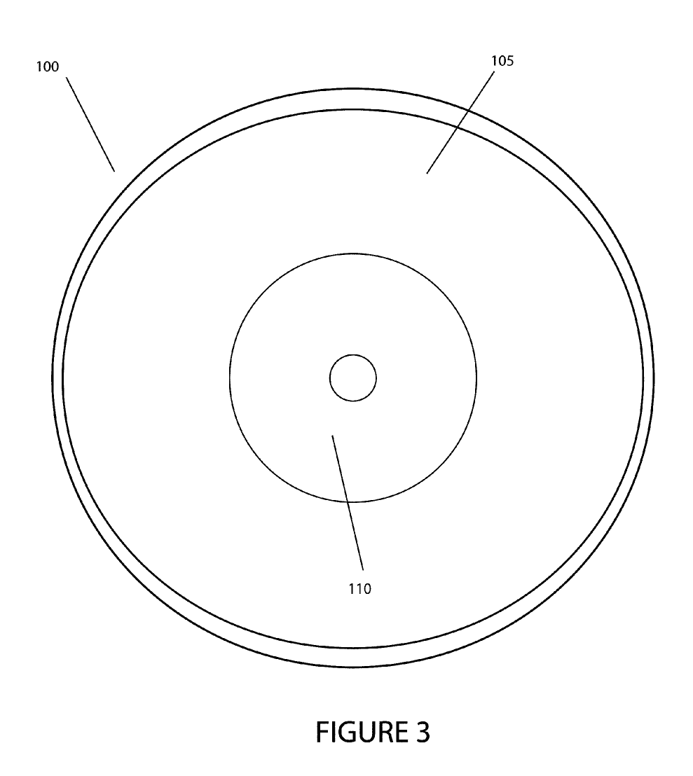

Referring to FIG. 1, the illustrated exemplary pad 100 includes a base 105 and a vertical target 110 in accordance with the principles of the invention. The vertical target 110 comprises an erect structure extending upwardly from the base 105. Importantly, the vertical target 110 should have a height that exceeds the spraying height of the animal. Additionally, the vertical target 110 should be slim relative to the base 105, such that the vertical target does not occupy most of the area of the base 105, as conceptually illustrated in FIG. 3. Animals require ample room around the vertical target 110 to urinate and defecate without spraying. Thus the amount of surrounding space of the base 105 should be adequate for a given animal.

Although the vertical target 110 is depicted as a conical projection, the invention is not limited to such a particular geometry of a vertical target 100. Other three dimensional shapes may be utilized within the scope of the invention. For example, vertical projections having circular, rectangular, square, triangular, pentagonal, octagonal or any other shape cross-section, whether it is uniform or non-uniform along the length of the vertical target 110, may be utilized within the spirit and scope of the invention.

While the base 105 is shown as being circular in FIGS. 1-3, the pad is not limited to a particular shape. Rectangular, triangular, oval, elliptical as well as other polygonal and alternative base 105 shapes may be utilized within the scope of the invention.

The base 105 should be sized to accommodate an animal, providing ample room for an animal to comfortably defecate and urinate thereon. The base should also be sized to provide adequate material to absorb a determined volume of urine expelled from an animal. Within these guidelines, the size of the base 105 may vary widely.

In a preferred embodiment, the vertical target 110 is located at or near the center of the pad 100. Central location makes the vertical target 110 attractive to animals because it provides accessibility from all sides of the vertical target 110. Thus an animal may instinctively sniff and spray the front, back and each side of (i.e., 360° around) the vertical target 110, at various possible angles. Thus, the vertical target 110 is configured for 360° accessibility. Central location also helps ensure that overspray (i.e., a portion of spray that misses the vertical target 110) and spattered spray will consistently land on the base 105.

The vertical target 110 may be an integral part of the pad 100 or a separate structure permanently (or releasably) attached to the base 105. Additionally, the vertical target may be solid or hollow, filled or unfilled.

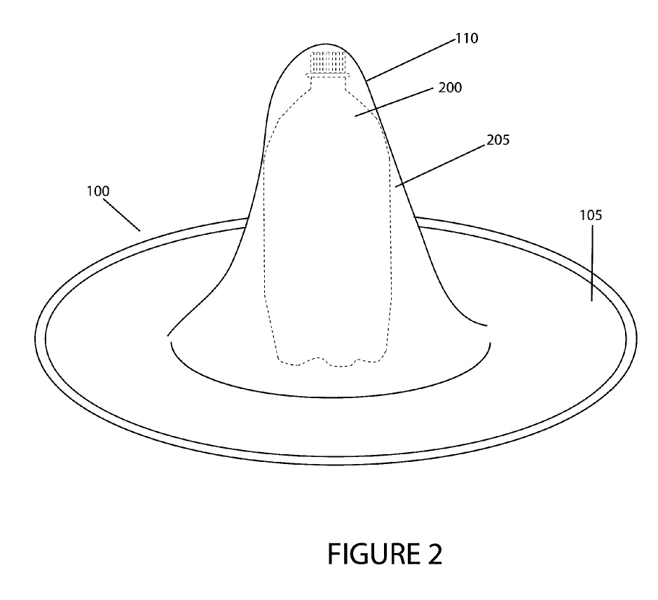

In a particular exemplary implementation, the vertical target 110 is comprised of a pocket 205 formed in the pad 100, as illustrated in FIG. 2. The pocket 205 may be formed by stitching or bonding material around an aperture in the base. Alternatively, the pocket 205 may be formed into the pad 100 using any technique compatible with the pad 100 material. By way of example, the pocket may be pressed or molded into the pad 100. These processes may be performed at elevated temperatures and pressures to permanently shape the pocket into the pad 100 material.

To maintain the pocketed vertical target 110 in an erect state, a supporting object 200 may be placed within the pocket 205. By way of example and not limitation, a plastic bottle 200, such as a two-liter soft drink bottle, may be utilized for support. The bottle 200 may be empty or partially or completely filled for additionally stability. Other objects shaped for supporting the vertical target 110 in an erect state may be utilized in lieu of a bottle. Illustratively, an inflatable bladder, plastic framework, foam rubber insert or other structure may be utilized to maintain the vertical target 110 in an erect state.

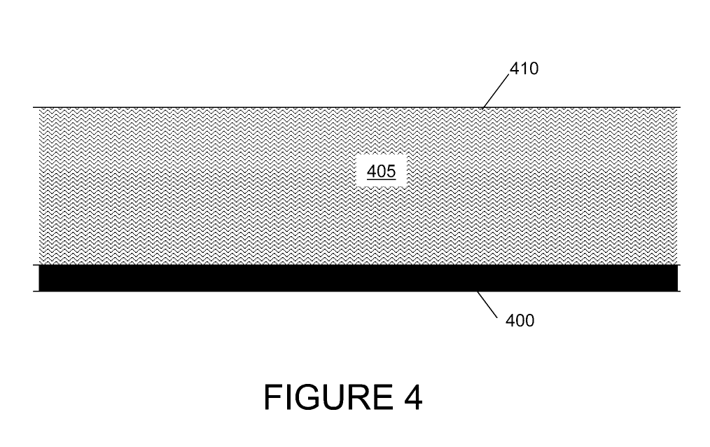

FIG. 4 representatively illustrates a cross section of the pad 100 of FIG. 1 taken along the line 4-4. The pad 100 (preferably including the vertical target 110, includes a substantially fluid impermeable backsheet layer 400 and an absorbent layer 405. Optionally, a topsheet layer 410 may be provided above the absorbent layer 405, so that the absorbent layer 405 is sandwiched between the backsheet layer 400 and the topsheet layer 410. The topsheet layer 410 is designed to be generally positioned toward the animal. Conversely, the backsheet layer 400 is designed to be generally positioned toward the floor. However, in various configurations, a portion of the backsheet layer 400 may be positioned towards the animal, or a portion of the topsheet layer 410 may be positioned towards the floor, or both, such as with folded or rolled edges.

The exemplary backsheet layer 400 may be made of a material that is formed or treated to be fluid impermeable. The backsheet layer 400 may be a single layer of material or a laminate of two or more separate layers of material. For example, the backsheet layer 400 may be a thin, substantially fluid impermeable web or sheet of plastic film such as polyethylene, polypropylene, polyolefin copolymers, combinations thereof or similar materials. One suitable material for the backsheet layer 400 is a 0.028 millimeter (mm) thick polyethylene film.

The exemplary absorbent layer 405 is made of materials adapted to absorb and retain urine, blood, or other body excrement. The absorbent layer 405 may contain various natural or synthetic absorbent materials, such as cellulose fibers, surfactant treated meltblown fibers, wood pulp fibers, regenerated cellulose or cotton fibers, a blend of pulp and other fiber, or the like, or combinations thereof. One such material is a coform material which is composed of a mixture of cellulosic fibers and synthetic polymer fibers. The absorbent layer 405 may also include compounds to increase its absorbency, such as 0-95 weight percent of organic or inorganic high-absorbency materials, commonly referred to as “superabsorbents”, which are typically capable of absorbing at least about 15 and desirably more than 25 times their weight in water. Suitable high-absorbency materials are described in U.S. Pat. No. 4,699,823 issued Oct. 13, 1987, to Kellenberger et al. and U.S. Pat. No. 5,147,343 issued Sep. 15, 1992, to Kellenberger, which are incorporated herein by reference. High-absorbency materials are available from various commercial vendors, such as Dow Chemical Company, Hoechst Celanese Corporation, and Allied Colloids, Inc.

The optional topsheet layer 410 is formed of a fluid permeable material so that fluid waste, and possibly semi-solid waste as well, can pass through and be absorbed by the absorbent layer 405. The topsheet layer 410 helps maintain integrity of the underlying absorbent layer 405. Suitable topsheet layers 410 may be made of a nonwoven web, a spunbond, meltblown or bonded-carded web composed of synthetic polymer filaments or fibers, such as polypropylene, polyethylene, polyesters or the like, a perforated film, or a web of natural polymer filaments or fibers such as rayon or cotton or combinations thereof. In addition, the topsheet layer 410 may be treated with a surfactant to aid in fluid transfer.

Various additives may be introduced into the pad to impart desired properties. For example, an odor attractant such as allyl phenyl acetate, and/or a deodorizer (e.g., cedar particles), and/or a disinfectant may be provided. These additives may be incorporated as pellets, powders, liquids or slurries into the pad 100 material.

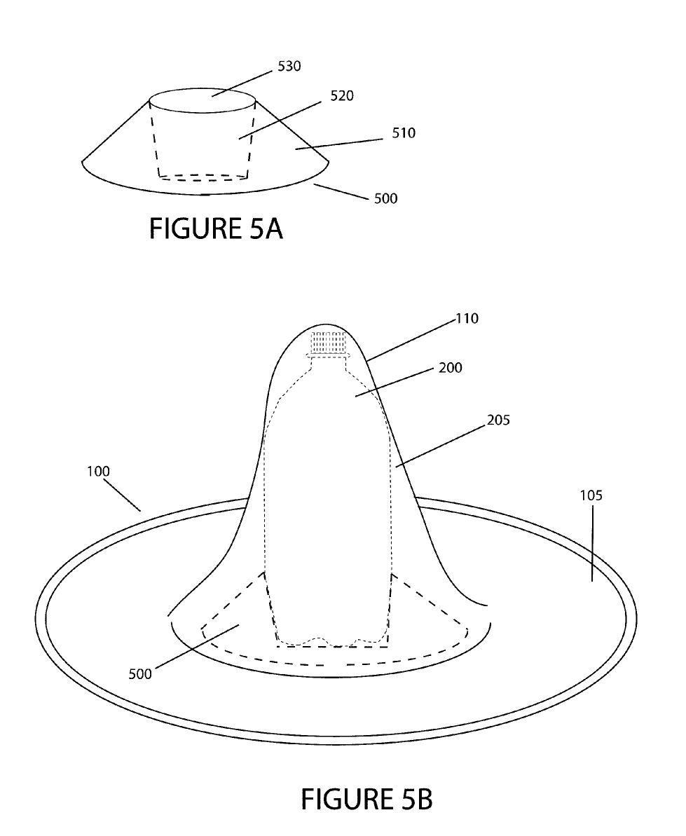

Referring now to FIGS. 5A and 5B, an optional support holder 500 provides a relatively broad and sturdy support base 510 for stabilizing and maintaining the vertical support structure 200 in an erect state. Thus, a pet will not easily topple the vertical support structure 200. The exemplary support holder 500 includes an opening 530 and receptacle 520 configured for receiving the bottom end of the vertical support structure 200. Other support holder configurations may be utilized within the scope of the invention. By way of example and not limitation, an annular collar with a plurality of supporting legs, similar to a Christmas tree stand, may be utilized as a support holder 500 within the scope of the invention.

While the invention has been described in terms of various embodiments, implementations and examples, those skilled in the art will recognize that the invention can be practiced with modification within the spirit and scope of the appended claims including equivalents thereof. The foregoing is considered as illustrative only of the principles of the invention. Variations and modifications may be affected within the scope and spirit of the invention.

1. An animal urine spray pad comprising a horizontal base and a vertical target attached to said base, said vertical target extending upwardly from the horizontal base and being configured for 360° accessibility, said base comprising a padded structure adapted for absorbing urine and being sized to provide room for an animal to comfortably defecate and urinate thereon said vertical target having a height and said height exceeding a urine spraying height of the animal.

2. An animal urine spray pad according to claim 1, wherein said base and vertical target are comprised of a laminate including an impermeable layer and an absorbent layer.

3. An animal urine spray pad according to claim 1, wherein said base and vertical target are comprised of a laminate including an impermeable layer, an absorbent layer and a permeable topsheet layer, said absorbent layer being disposed between said impermeable and permeable topsheet layers.

4. An animal urine spray pad according to claim 1, wherein said vertical target is comprised of a pocket attached to said horizontal base.

5. An animal urine spray pad according to claim 1, wherein said vertical target is comprised of a pocket formed in said horizontal base.

6. An animal urine spray pad according to claim 1, wherein said vertical target is comprised of a pocket attached to said horizontal base and a support object disposed within said pocket, said support object being configured to maintain the pocket erect.

7. An animal urine spray pad according to claim 1, wherein said vertical target is comprised of a pocket formed in said horizontal base and a support object disposed within said pocket, said support object being configured to maintain the pocket erect.

8. An animal urine spray pad according to claim 1, wherein said vertical target includes a support object being configured to maintain the pocket erect, said support object comprising a plastic bottle.

9. An animal urine spray pad according to claim 1, wherein said vertical target is removably attached to said horizontal base.

10. An animal urine spray pad according to claim 1, wherein said vertical target is permanently attached to said horizontal base.

11. An animal urine spray pad according to claim 1, wherein said vertical target is attached to the horizontal base near the center of the horizontal base.

12. An animal urine spray pad comprising a horizontal base and a vertical target formed in said base, said vertical target extending upwardly from the horizontal base and being configured for 360° accessibility, said base comprising a padded structure adapted for absorbing urine and being sized to provide room for an animal to comfortably defecate and urinate thereon, said vertical target having a height and said height exceeding a urine spraying height of the animal.

13. An animal urine spray pad according to claim 12, wherein said base and vertical target are comprised of a laminate including an impermeable layer and an absorbent layer.

14. An animal urine spray pad according to claim 12, wherein said base and vertical target are comprised of a laminate including an impermeable layer, an absorbent layer and a permeable topsheet layer, said absorbent layer being disposed between said impermeable and permeable topsheet layers.

15. An animal urine spray pad according to claim 12, wherein said vertical target is comprised of a pocket attached to said horizontal base.

16. An animal urine spray pad according to claim 12, wherein said vertical target is comprised of a pocket formed in said horizontal base.

17. An animal urine spray pad according to claim 12, wherein said vertical target is comprised of a pocket formed in said horizontal base and a support object disposed within said pocket, said support object being configured to maintain the pocket erect.

18. An animal urine spray pad according to claim 17, wherein said vertical target is further comprised of a holder configured to engage the support object and maintain the support object erect.

19. An animal urine spray pad according to claim 1, wherein said vertical target is removably attached to said horizontal base.

20. An animal urine spray pad according to claim 1, wherein said vertical target is attached to the horizontal base near the center of the horizontal base.
